(19) United States
(12) Patent Application Publication
(10) Pub. No.: US 2015/0192559 A1
(43) Pub. Date: Jul. 9, 2015

(54) DIAGNOSIS METHOD AND MAINTENANCE METHOD FOR OIL-FILLED ELECTRICAL EQUIPMENT

(71) Applicants: Fukutaro Kato, Chiyoda-ku (JP); Tsuyoshi Amimoto, Chiyoda-ku (JP); Ryuichi Nishiura, Chiyoda-ku (JP); Satoru Toyama, Chiyoda-ku (JP); Kota Mizuno, Chiyoda-ku (JP)

(72) Inventors: Fukutaro Kato, Chiyoda-ku (JP); Tsuyoshi Amimoto, Chiyoda-ku (JP); Ryuichi Nishiura, Chiyoda-ku (JP); Satoru Toyama, Chiyoda-ku (JP); Kota Mizuno, Chiyoda-ku (JP)

(73) Assignee: Mitsubishi Electric Corporation, Chiyoda-ku (JP)

(21) Appl. No.: 14/417,472

(22) PCT Filed: Nov. 20, 2012
(86) PCT No.: PCT/JP2012/080053

§ 371 Date: Jan. 26, 2015

(51) Int. Cl. G01N 33/28; G01M 99/00

(52) U.S. Cl. CPC G01N 33/287; G01M 99/008

(57) ABSTRACT

A diagnosis method for oil-filled electrical equipment, for diagnosing a risk of occurrence of an abnormality caused by generation of copper sulfide on insulating paper, wherein a step 1 of conducting sulfidation corrosion evaluation of insulating oil in the oil-filled electrical equipment is performed, any one of a step 2A-1 of analyzing the insulating oil for presence of dibenzyldisulfide and an oxidative degradation preventing agent, a step 2A-2 of analyzing the insulating oil for presence of a copper sulfide generation inhibitor, and a step 2B of checking presence of oxygen in an atmosphere of the insulating oil is performed, a step 3 of analyzing the insulating oil for presence of a byproduct derived when copper sulfide is generated from dibenzyldisulfide is performed, and a step 4 of diagnosing the risk of occurrence of an abnormality based on results of the steps 1, 2A-1, 2A-2, and 3 is performed.

### TECHNICAL FIELD

The present invention relates to a method for diagnosing the risk of occurrence of an abnormality in oil-filled electrical equipment caused by generation of copper sulfide on a surface of insulating paper provided on coil copper in the oil-filled electrical equipment, and a maintenance method for oil-filled electrical equipment.

### BACKGROUND ART

In oil-filled electrical equipment such as a transformer, coil copper as a conducting medium has insulating paper wound therearound so that a structure for preventing occurrence of short-circuit between adjoined turns is provided.

However, mineral oil (insulating oil) used in the transformer contains a sulfur constituent, and the sulfur constituent reacts with coil copper arranged in oil, so that conductive copper sulfide is generated. In the case where this copper sulfide is generated on a surface of insulating paper provided on the coil, a conduction path is formed from a point at which copper sulfide is deposited because copper sulfide is a conductive substance. Consequently, there has been known disadvantages such as occurrence of electric breakdown due to short-circuit of adjoining coil turns.

Moreover, it has been known that a causative substance causing generation of copper sulfide is dibenzyldisulfide (DBDS) which is a kind of a sulfur compound in oil. It has also been known that copper sulfide is generated on coil insulating paper by a process in which dibenzyldisulfide reacts with coil copper to generate a complex, a process in which the complex is diffused in oil to adhere to coil insulating paper, and a process in which the adhered complex is dissolved to become copper sulfide (e.g., PTD 1: Japanese Patent Laying-Open No. 2011-165851).

FIG. 3 shows a mechanism of generation of copper sulfide in oil-filled electrical equipment under the oxygen-free atmosphere. As shown in FIG. 3, the copper sulfide generation reaction is divided into two stages. In a first stage, copper and DBDS react chemically to generate a copper-DBDS complex (intermediate substance). This complex diffuses in insulating oil and a part thereof adheres to insulating paper. In a second stage, the aforementioned complex is dissolved by thermal energy, and thereby, copper sulfide is deposited on the insulating paper.

Furthermore, according to the aforementioned generation mechanism, by suppressing the reaction between dibenzyldisulfide and coil copper, copper sulfide generation can be suppressed. When 1,2,3-benzotriazole (BTA) or Irgamet 39 is, for example, added into the insulating oil as a copper sulfide generation inhibitor, the inhibitor reacts with coil copper to form a film on a coil copper surface. It has been known that this formed film blocks and suppresses the reaction between dibenzyldisulfide and coil copper, and thus, copper sulfide generation can be suppressed (e.g., NPD 1 (T. Amimoto, E. Nagao, J. Tanimura, S. Toyama and N. Yamada, “Duration and Mechanism for Suppressive Effect of Triazole-based Passivators on Copper-sulfide Deposition on Insulating Paper”, IEEE Transactions on Dielectrics and Electrical Insulation, Vol. 16, No. 1, pp. 257-264, 2009.)).

On the other hand, the insulating oil used in the oil-filled electrical equipment such as a transformer is generally large in amount and long in age of service, and thus, replacement is not easy. Therefore, in each oil-filled electrical equipment in which the insulating oil containing the sulfur constituent is used, there is a demand to predict occurrence of an abnormality such as electric breakdown caused by deposition of copper sulfide and take necessary measures at the right time.

Conventionally, the risk of such occurrence of an abnormality in the oil-filled electrical equipment has been evaluated based on the analysis of dibenzyldisulfide in the insulating oil and the sulfidation corrosion test (such as IEC62535) of the insulating oil.

However, in the oil-filled electrical equipment, copper sulfide is generated not only on the coil insulating paper but also on coil copper, PB (pressboard) and the like, and the risk of occurrence of an abnormality such as electric breakdown varies depending on sites of copper sulfide generation. Therefore, it is conceivable that the risk of occurrence of an abnormality in the oil-filled electrical equipment cannot necessarily be evaluated even if the possibility of copper sulfide generation is predicted simply by measuring a causative substance such as dibenzyldisulfide.

In addition, from recent study results, it has been found that oxygen and an oxidative degradation preventing agent (such as 2,6-di-t-butyl-p-cresol) dissolved in the insulating oil are factors for accelerating copper sulfide generation (e.g., NPD 2 (S. Toyama, K. Mizuno, F. Kato, E. Nagao, T. Amimoto, and N. Hosokawa, “Influence of Inhibitor and Oil Components on Copper Sulfide Deposition on Kraft Paper in Oil-immersed Insulation”, IEEE Transactions on Dielectrics and Electrical Insulation, Vol. 18, No. 6, pp. 1877-1885, 2011.), NPD 3 (H. Kawarai, Y. Fujita, J. Tanimura, S. Toyama, N. Yamada, E. Nagao, N. Hosokawa and T. Amimoto, “Role of Dissolved Copper and Oxygen on Copper Sulfide Generation in Insulating Oil”, IEEE Transactions on Dielectrics and Electrical Insulation, Vol. 16, No. 5, pp. 1430-1435, 2009.)).

Furthermore, when the aforementioned copper sulfide generation inhibitor such as Irgamet 39 or 1,2,3-benzotriazole (BTA) is added into the insulating oil, it is also considered to be necessary to take into consideration the fact that this inhibitor becomes a factor for suppressing copper sulfide generation.

For these reasons, there has been a possibility that the risk of occurrence of an abnormality in the oil-filled electrical equipment cannot be accurately evaluated only by the conventional analysis of dibenzyldisulfide in the insulating oil and the conventional sulfidation corrosion test.

### CITATION LIST

### Patent Document

PTD 1: Japanese Patent Laying-Open No. 2011-165851

### Non Patent Document

NPD 1: T. Amimoto, E. Nagao, J. Tanimura, S. Toyama and N. Yamada, “Duration and Mechanism for Suppressive Effect of Triazole-based Passivators on Copper-sulfide Deposition on Insulating Paper”, IEEE Transactions on Dielectrics and Electrical Insulation, Vol. 16, No. 1, pp. 257-264, 2009.

NPD 2: S. Toyama, K. Mizuno, F. Kato, E. Nagao, T. Amimoto, and N. Hosokawa, “Influence of Inhibitor and Oil Components on Copper Sulfide Deposition on Kraft Paper in Oil-immersed Insulation”, IEEE Transactions on Dielectrics and Electrical Insulation, Vol. 18, No. 6, pp. 1877-1885, 2011.

NPD 3: H. Kawarai, Y. Fujita, J. Tanimura, S. Toyama, N. Yamada, E. Nagao, N. Hosokawa and T. Amimoto, “Role of Dissolved Copper and Oxygen on Copper Sulfide Generation in Insulating Oil”, IEEE Transactions on Dielectrics and Electrical Insulation, Vol. 16, No. 5, pp. 1430-1435, 2009.

### SUMMARY OF INVENTION

### Technical Problem

The present invention has been made to solve the aforementioned problem, and an object thereof is to provide a diagnosis method for oil-filled electrical equipment in which the risk of occurrence of an abnormality caused by generation of copper sulfide on insulating paper in the oil-filled electrical equipment can be diagnosed with high precision, and a more appropriate maintenance method for the oil-filled electrical equipment based on the diagnosis result.

### Solution to Problem

The present invention is directed to a diagnosis method for oil-filled electrical equipment, for diagnosing a risk of occurrence of an abnormality caused by generation of copper sulfide on insulating paper in the oil-filled electrical equipment, wherein

(1) a step 1 of conducting sulfidation corrosion evaluation of insulating oil in the oil-filled electrical equipment is performed,

(2A-1) when the insulating oil is evaluated as being non-corrosive in the step 1, a step 2A-1 of analyzing the insulating oil for presence or absence of dibenzyldisulfide and an oxidative degradation preventing agent is performed,

(2A-2) when the dibenzyldisulfide and the oxidative degradation preventing agent are both substantially detected in the insulating oil in the step 2A-1, a step 2A-2 of analyzing the insulating oil for presence or absence of a copper sulfide generation inhibitor is further performed,

(2B) when the insulating oil is evaluated as being corrosive in the step 1, a step 2B of checking presence or absence of oxygen in an atmosphere of the insulating oil is performed,

(3) when the insulating oil is evaluated as being corrosive in the step 1 and when at least one of the dibenzyldisulfide and the oxidative degradation preventing agent is not substantially detected in the insulating oil in the step 2A-1, a step 3 of analyzing the insulating oil for presence or absence of a byproduct derived when copper sulfide is generated from dibenzyldisulfide is further performed, and

(4) a step 4 of diagnosing the risk of occurrence of an abnormality based on results of all steps performed, of the steps 1, 2A-1, 2A-2, and 3, is performed.

Preferably, the oxidative degradation preventing agent is 2,6-di-t-butyl-p-cresol.

Preferably, the copper sulfide generation inhibitor is a benzotriazole compound.

Preferably, in the step 2B, presence or absence of oxygen in the atmosphere of the insulating oil is checked by checking whether the oil-filled electrical equipment is of open type or of sealed type.

Preferably, the byproduct is at least one kind of compound selected from a group consisting of benzaldehyde, benzyl alcohol, bibenzyl, dibenzyl sulfide, and dibenzyl sulphoxide.

The present invention also relates to a maintenance method for oil-filled electrical equipment, for taking prescribed measures in accordance with the risk, based on the risk diagnosed by using the aforementioned diagnosis method for oil-filled electrical equipment.

Preferably, the measures include at least any one of addition of the copper sulfide generation inhibitor, recommendation of an oil degradation preventing method for the oil-filled electrical equipment, replacement with new oil that does not contain dibenzyldisulfide, and update of the insulating oil.

### ADVANTAGEOUS EFFECTS OF INVENTION

In the diagnosis method for the oil-filled electrical equipment according to the present invention, the other factors for accelerating and suppressing copper sulfide generation are incorporated into items of determination, in addition to the conventional analysis of dibenzyldisulfide in the insulating oil and the conventional sulfidation corrosion evaluation of the insulating oil. Therefore, the risk of occurrence of an abnormality in the oil-filled electrical equipment caused by generation of copper sulfide on the surface of the insulating paper provided on coil copper in the oil-filled electrical equipment can be diagnosed with high precision. In addition, more appropriate maintenance of the oil-filled electrical equipment can be performed based on the diagnosis result.

### BRIEF DESCRIPTION OF DRAWINGS

FIG. 1 is a flowchart for describing one example of a diagnosis method for oil-filled electrical equipment according to the present invention.

FIG. 2 is a flowchart for describing one example of a maintenance method for the oil-filled electrical equipment according to the present invention.

FIG. 3 is a schematic view for describing a mechanism of generation of copper sulfide in the oil-filled electrical equipment.

### DESCRIPTION OF EMBODIMENTS

(Diagnosis Method for Oil-Filled Electrical Equipment)

The present invention is directed to a diagnosis method for oil-filled electrical equipment, for diagnosing the risk of occurrence of an abnormality caused by generation of copper sulfide on insulating paper in the oil-filled electrical equipment. The diagnosis method for the oil-filled electrical equipment according to the present invention is characterized in that not only the results of the conventional analysis of dibenzyldisulfide in the insulating oil and the conventional sulfidation corrosion test but also the prescribed factors for accelerating and suppressing copper sulfide generation are incorporated into items of determination, depending on an object to be diagnosed.

Specifically, in the diagnosis method according to the present invention,

(1) a step 1 of conducting sulfidation corrosion evaluation of insulating oil in the oil-filled electrical equipment is performed,

(2A-1) when the insulating oil is evaluated as being non-corrosive in the step 1, a step 2A-1 of analyzing the insulating oil for presence or absence of dibenzyldisulfide and an oxidative degradation preventing agent is performed,

(2A-2) when the dibenzyldisulfide and the oxidative degradation preventing agent are both substantially detected in the insulating oil in the step 2A-1, a step 2A-2 of analyzing the insulating oil for presence or absence of a copper sulfide generation inhibitor is further performed,

(2B) when the insulating oil is evaluated as being corrosive in the step 1, a step 2B of checking presence or absence of oxygen in an atmosphere of the insulating oil is performed,

(3) when the insulating oil is evaluated as being corrosive in the step 1 and when at least one of the dibenzyldisulfide and the oxidative degradation preventing agent is not substantially detected in the insulating oil in the step 2A-1, a step 3 of analyzing the insulating oil for presence or absence of a byproduct derived when copper sulfide is generated from dibenzyldisulfide is further performed, and

(4) a step 4 of diagnosing the risk of occurrence of an abnormality based on results of all steps performed, of the steps 1, 2A-1, 2A-2, and 3, is performed.

“Sulfidation corrosion evaluation” herein refers to evaluating the sulfidation corrosion of the insulating oil with respect to copper and evaluating the risk of copper sulfide generation on the insulating paper at and after the evaluation time point, by using a prescribed sulfidation corrosion test. The sulfidation corrosion test method used in sulfidation corrosion evaluation includes, for example, a test method according to a sulfidation corrosion test based on IEC (IEC62535) and a test method according to a sulfidation corrosion test based on ASTM (ASTM D 1275B).

In the normal IEC62535 sulfidation corrosion test and the like, if copper sulfide is detected on any one of coil copper and the insulating paper after the test, the insulating oil is evaluated as being corrosive. However, electric breakdown due to short-circuit of coil turns is a phenomenon caused by generation of copper sulfide on the insulating paper. Therefore, in the present invention, the insulating oil is evaluated as being corrosive, only when copper sulfide is detected “on the insulating paper” after the test. Namely, even if copper sulfide is detected on coil copper, the insulating oil is evaluated as being non-corrosive when copper sulfide is not detected on the insulating paper. In terms of this point, the sulfidation corrosion test method used in sulfidation corrosion evaluation in the present invention is different from IEC62535.

“Analyzing the insulating oil” refers to, for example, measuring the compounds (dibenzyldisulfide, the oxidative degradation preventing agent, the copper sulfide generation inhibitor, and the byproduct derived when copper sulfide is generated from dibenzyldisulfide) in the insulating oil, and detecting the presence or absence of each compound in the insulating oil. However, the presence or absence of DBPC in the insulating oil may be determined from, for example, a brand of the used insulating oil and the like, and the case of not conducting actual measurement as described above is also included in the aforementioned “analyzing”.

Each compound in the insulating oil can be detected by using the existing technique. By using, for example, a measuring instrument such as a gas chromatograph/mass spectrometer, HPLC (high-performance liquid chromatography), an amount of each compound can be detected up to around 1 ppmw.

In the oil-filled electrical equipment such as a transformer, it is difficult to inspect the insulating paper portion of the coil during operation. However, the diagnosis method according to the present invention has an advantage that the possibility of copper sulfide generation on the insulating paper can be evaluated with high precision, based on constituent analysis and the like of the insulating oil extracted from the oil-filled electrical equipment.

The aforementioned diagnosis method according to the present invention is mainly formed of the following four-stage steps:

(1) a step 1 of conducting sulfidation corrosion evaluation of insulating oil in the oil-filled electrical equipment;

(2) at least any one of a step 2A-1 of analyzing the insulating oil for presence or absence of dibenzyldisulfide and an oxidative degradation preventing agent,

a step 2A-2 of analyzing the insulating oil for presence or absence of a copper sulfide generation inhibitor, and

a step 2B of checking presence or absence of oxygen in an atmosphere of the insulating oil;

(3) a step 3 of analyzing, as necessary, the insulating oil for presence or absence of a byproduct derived when copper sulfide is generated from dibenzyldisulfide; and

(4) a step 4 of diagnosing the risk of occurrence of an abnormality based on results of all steps performed, of the steps 1, 2A-1, 2A-2, and 3.

Namely, in (1) step 1, the possibility of copper sulfide generation in the future is roughly evaluated.

In (2) step 2A-1, step 2A-2 and step 2B, the possibility of copper sulfide generation in the future is evaluated in more detail.

In (3) step 3, the possibility of copper sulfide generation at the present moment is evaluated.

In (4) step 4, in the diagnosis method according to the present invention, the risk of occurrence of an abnormality in the oil-filled electrical equipment caused by generation of copper sulfide on the surface of the insulating paper provided on coil copper in the oil-filled electrical equipment is diagnosed based on the results of all steps performed.

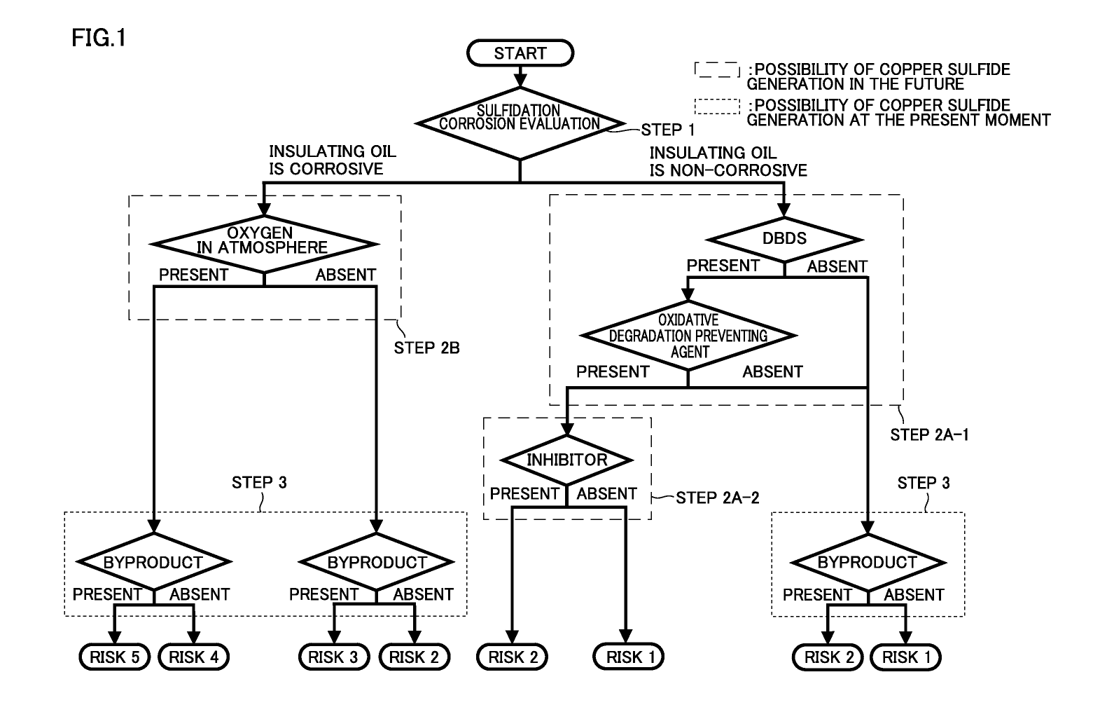

Next, one example of the diagnosis method for the oil-filled electrical equipment according to the present invention will be described with reference to FIG. 1.

(Step 1)

First, the sulfidation corrosion of the insulating oil is evaluated by the sulfidation corrosion test according to IEC62535, and the risk of copper sulfide generation on the insulating paper at and after the evaluation time point is determined. IEC62535 is a test in which a test piece of coil copper and the insulating paper is immersed in the insulating oil, is heated to a prescribed temperature in the air atmosphere and stored for a prescribed time, and thereafter, copper sulfide generation on the test piece is observed. As described above, however, in the present invention, the insulating oil is evaluated as being corrosive only when copper sulfide is detected on the insulating paper after the test, and even if copper sulfide is detected on coil copper, the insulating oil is evaluated as being non-corrosive when copper sulfide is not detected on the insulating paper.

In the IEC62535 sulfidation corrosion test, the insulating oil is exposed to the air atmosphere, and thus, it is conceivable that oxygen is dissolved in the insulating oil. Therefore, when copper sulfide generation is detected on the insulating paper after the test, it is considered to be highly likely that dibenzyldisulfide and 2,6-di-t-butyl-p-cresol (DBPC) are both present in the insulating oil.

<Step 2A-1, Step 2A-2, Step 2B>

The possibility of copper sulfide generation in the future is the highest when DBDS, DBPC and oxygen are all present in the insulating oil. This is because it has been known that DBPC and oxygen are factors for accelerating copper sulfide generation on the insulating paper (e.g., NPD 2 and NPD 3).

Therefore, when DBDS and DBPC are contained in the insulating oil and the concentration of oxygen in the insulating oil is high (e.g., when the transformer is of open type), the risk of electric breakdown due to copper sulfide generation is considered to be extremely high. On the other hand, when the concentration of oxygen in the insulating oil is low (e.g., when the transformer is of sealed type) even if DBDS is contained in the insulating oil, or when DBPC is not contained in the insulating oil, copper sulfide generation on the insulating paper is unlikely and the risk of occurrence of an abnormality caused by copper sulfide generation is considered to be extremely low.

When DBDS is not contained in the insulating oil, the possibility of copper sulfide generation at and after diagnosis is considered to be little.

Based on the foregoing consideration, evaluations in step 2A-1, step 2A-2 and step 2B are conducted in accordance with the flowchart shown in FIG. 1.

(Step 2A-1)

As shown in FIG. 1, when the insulating oil is evaluated as being non-corrosive in step 1 described above, step 2A-1 of analyzing the insulating oil for the presence or absence of dibenzyldisulfide and an oxidative degradation preventing agent is performed. The oxidative degradation preventing agent is preferably 2,6-di-t-butyl-p-cresol.

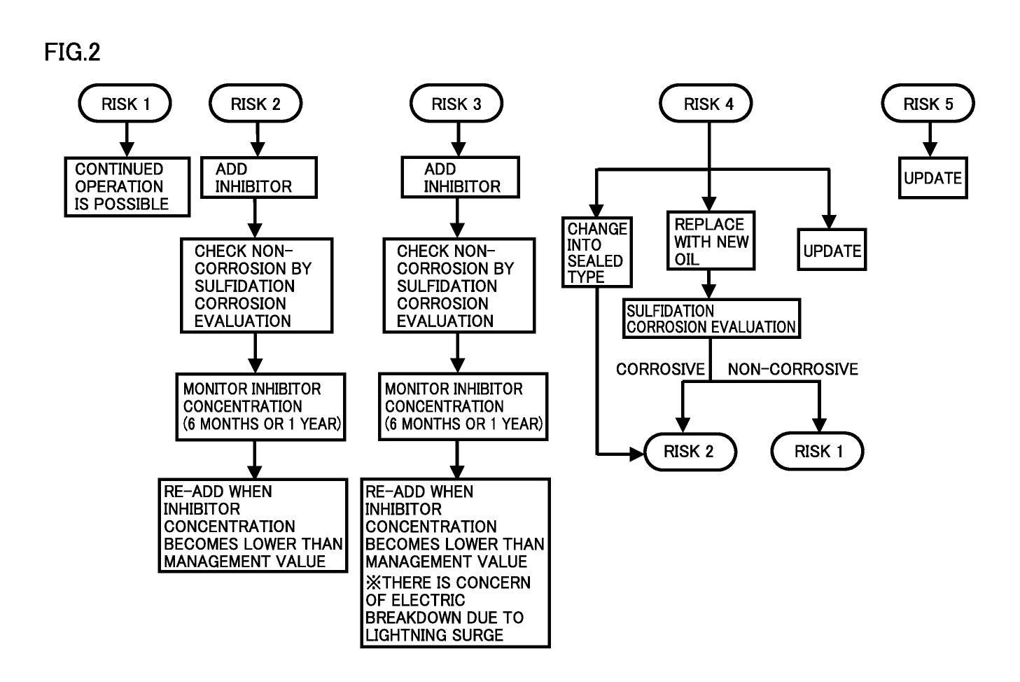

When either DBDS or DBPC is not contained in the insulating oil, the risk of occurrence of an abnormality is diagnosed as risk 1 or risk 2 after step 3 described below is performed. In FIGS. 1 and 2, “risk 1” means that the risk is the lowest, and “risk 5” means that the risk is the highest.

(Step 2A-2)

When the dibenzyldisulfide and the oxidative degradation preventing agent are both substantially detected in the insulating oil in step 2A-1 described above, step 2A-2 of analyzing the insulating oil for the presence or absence of a copper sulfide generation inhibitor is further performed.

When the copper sulfide generation inhibitor is contained in the insulating oil in step 2A-2, the risk of occurrence of an abnormality is diagnosed as risk 2. On the other hand, when the copper sulfide generation inhibitor is not contained in the insulating oil in step 2A-2, the risk of occurrence of an abnormality is diagnosed as risk 1.

The copper sulfide generation inhibitor to be analyzed in step 2A-2 is preferably a benzotriazole compound. The benzotriazole compound includes, for example, 1,2,3-benzotriazole (BTA), Irgamet (registered trademark) 39 [N,N-bis(2-ethylhexyl)-(4 or 5)-methyl-1H-benzotriazole-1-methylamine: manufactured by BASF Japan Ltd.].

(Step 2B)

On the other hand, when the insulating oil is evaluated as being corrosive in step 1, step 2B of checking the presence or absence of oxygen in the atmosphere of the insulating oil is next performed. A method for checking the presence or absence of oxygen in the atmosphere of the insulating oil includes, for example, a method for checking whether a type of the oil-filled electrical equipment is open type or sealed type. The method for checking the presence or absence of oxygen in the atmosphere of the insulating oil is not limited thereto, and a method for actually measuring oxygen in the atmosphere of the insulating oil, and other methods may be used.

When the oil-filled electrical equipment is of open type in which the concentration of oxygen in the insulating oil is generally high, the possibility of copper sulfide generation is high due to a synergistic effect between DBDS, DBPC and oxygen. On the other hand, when the oil-filled electrical equipment is of sealed type in which the concentration of oxygen in the insulating oil is generally low, the possibility of copper sulfide generation is considered to be lower than that in the open type oil-filled electrical equipment.

Based on the foregoing consideration, the risk of occurrence of an abnormality is determined as risks 2 to 5 after next step 3 is performed, in accordance with the flowchart shown in FIG. 1.

(Step 3)

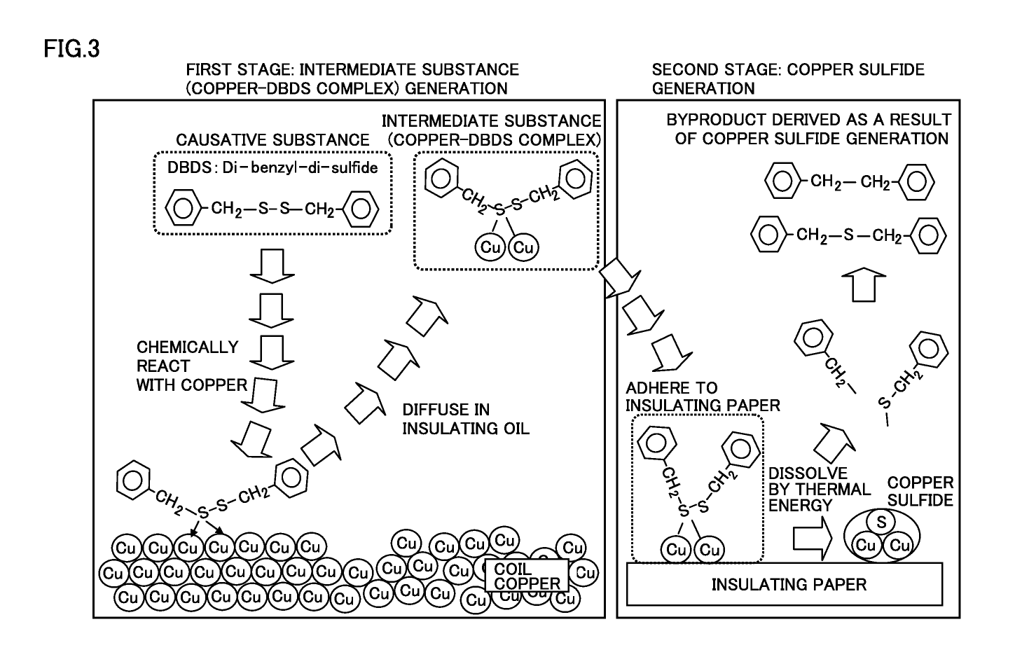

As step 3, the presence or absence of copper sulfide generation at the present moment (at the diagnosis time point) is evaluated. In step 3, false evaluation may be conducted if the possibility of copper sulfide generation is evaluated based only on an amount of DBDS, because DBDS is used and decreases as copper sulfide generation progresses (see FIG. 3). Therefore, it is preferable to evaluate the possibility of copper sulfide generation by using, as indicators, not only DBDS but also a byproduct (trace of DBDS) derived when copper sulfide is generated from DBDS. From a result of analysis of the insulating oil for the presence or absence of the byproduct at the time of copper sulfide generation, the presence or absence of copper sulfide generation at the present moment can be evaluated. When the byproduct is detected, it is conceivable that copper sulfide is generated at the present moment. On the other hand, when the byproduct is not detected, the possibility of copper sulfide generation at the present moment is considered to be low.

The byproduct at the time of copper sulfide generation is preferably at least one kind of compound selected from the group consisting of benzaldehyde, benzyl alcohol, bibenzyl, dibenzyl sulfide, and dibenzyl sulphoxide.

(Step 4)

Next, based on the results of all steps performed, of steps 1, 2A-1, 2A-2, and 3 described above, the risk of occurrence of an abnormality in the oil-filled electrical equipment is diagnosed in accordance with the flowchart shown in FIG. 1. In such a manner, the risk of occurrence of an abnormality in the oil-filled electrical equipment is comprehensively diagnosed based on the results of the aforementioned steps as to the presence or absence of copper sulfide generation in the future and the presence or absence of copper sulfide generation at the present moment.

(Maintenance Method for Oil-Filled Electrical Equipment)

In a maintenance method for the oil-filled electrical equipment according to the present invention, prescribed measures (maintenance) are taken in accordance with the risk, based on the risk of occurrence of an abnormality diagnosed by using the aforementioned diagnosis method for the oil-filled electrical equipment.

The aforementioned “measures” preferably include at least any one of addition of the copper sulfide generation inhibitor, recommendation of an oil degradation preventing method for the oil-filled electrical equipment, replacement with new oil that does not contain dibenzyldisulfide (causative substance), and update of the insulating oil.

One example of the maintenance method for the oil-filled electrical equipment based on the result of diagnosis conducted in accordance with the flowchart shown in FIG. 1 above will be described below with reference to FIG. 2.

First, when the risk of occurrence of an abnormality is diagnosed as risk 1, continued operation is possible, and thus, no measures are particularly taken.

When the risk of occurrence of an abnormality is diagnosed as risk 2, the copper sulfide generation inhibitor is added. It has been known that addition of the copper sulfide generation inhibitor to the insulating oil makes it possible to suppress copper sulfide generation on the coil insulating paper (e.g., NPD 1). Namely, by addition of the copper sulfide generation inhibitor, a film is formed on the coil copper surface and this film suppresses the reaction between dibenzyldisulfide and coil copper, and thus, copper sulfide generation is suppressed. The copper sulfide generation inhibitor includes Irgamet 39 and BTA.

After the copper sulfide generation inhibitor is added, it is further checked by sulfidation corrosion evaluation that the insulating oil is non-corrosive, and the concentration of the copper sulfide generation inhibitor is monitored. Monitoring is performed every six months or every one year, for example. When the concentration of the copper sulfide generation inhibitor becomes lower than a prescribed management value (e.g., 1 ppm) as a result of monitoring, the copper sulfide generation inhibitor is re-added.

When the risk of occurrence of an abnormality is diagnosed as risk 3, the copper sulfide generation inhibitor is added, and it is checked by sulfidation corrosion evaluation that the insulating oil is non-corrosive, and the concentration of the copper sulfide generation inhibitor is monitored, and when the concentration of the copper sulfide generation inhibitor becomes lower than the prescribed management value, the copper sulfide generation inhibitor is re-added, similarly to the case in which the risk of occurrence of an abnormality is diagnosed as risk 2. However, when the risk of occurrence of an abnormality is diagnosed as risk 3, an insulation distance between adjoining coil turns may be short because copper sulfide is generated at the diagnosis time point. Therefore, there is concern of electric breakdown due to lightning surge.

When the risk of occurrence of an abnormality is diagnosed as risk 4, any one of change of the type of the oil-filled electrical equipment into sealed type, replacement of the insulating oil with new oil, and update into newly established equipment is performed. When the change of the type of the oil-filled electrical equipment into sealed type is performed, the measures similar to those taken in the aforementioned case where the risk of occurrence of an abnormality is diagnosed as risk 2 are further taken. When the replacement of the insulating oil with new oil is performed, sulfidation corrosion evaluation of the new oil is further conducted. When the new oil is non-corrosive, the measures similar to those taken in the aforementioned case where the risk of occurrence of an abnormality is diagnosed as risk 1 are taken. When the new oil is corrosive, the measures similar to those taken in the aforementioned case where the risk of occurrence of an abnormality is diagnosed as risk 2 are taken.

When the type of the oil-filled electrical equipment is open type, copper sulfide generation can be suppressed by changing the type into sealed type. In this case, an amount of oxygen transmitting through the insulating oil is generally small in the sealed type, and thus, copper sulfide generation is further suppressed as compared with the open type, even if DBDS and DBPC are contained in the insulating oil. A specific method for changing the type into sealed type includes a method for changing, into sealed type, a type of a conservator which is one component of the transformer, and other methods.

When the risk of occurrence of an abnormality is diagnosed as risk 5, update into newly established equipment is performed.

It should be understood that the embodiment disclosed herein is illustrative and not limitative in any respect. The scope of the present invention is defined by the terms of the claims, rather than the description above, and is intended to include any modifications within the scope and meaning equivalent to the terms of the claims.

1. A diagnosis method for oil-filled electrical equipment, for diagnosing a risk of occurrence of an abnormality caused by generation of copper sulfide on insulating paper in the oil-filled electrical equipment, wherein
(1) a step 1 of conducting sulfidation corrosion evaluation of insulating oil in said oil-filled electrical equipment is performed,
(2A-1) when said insulating oil is evaluated as being non-corrosive in said step 1, a step 2A-1 of analyzing said insulating oil for presence or absence of dibenzyldisulfide and an oxidative degradation preventing agent is performed,
(2A-2) when said dibenzyldisulfide and said oxidative degradation preventing agent are both substantially detected in said insulating oil in said step 2A-1, a step 2A-2 of analyzing said insulating oil for presence or absence of a copper sulfide generation inhibitor is further performed,
(2B) when said insulating oil is evaluated as being corrosive in said step 1, a step 2B of checking presence or absence of oxygen in an atmosphere of said insulating oil is performed,
(3) when said insulating oil is evaluated as being corrosive in said step 1 and when at least one of said dibenzyldisulfide and said oxidative degradation preventing agent is not substantially detected in said insulating oil in said step 2A-1, a step 3 of analyzing said insulating oil for presence or absence of a byproduct derived when copper sulfide is generated from dibenzyldisulfide is further performed, and
(4) a step 4 of diagnosing said risk of occurrence of an abnormality based on results of all steps performed, of said steps 1, 2A-1, 2A-2, and 3, is performed.

2. The diagnosis method according to claim 1, wherein said oxidative degradation preventing agent is 2,6-di-t-butyl-p-cresol.

3. The diagnosis method according to claim 1, wherein said copper sulfide generation inhibitor is a benzotriazole compound.

4. The diagnosis method according to any claim 1, wherein
in said step 2B, presence or absence of oxygen in the atmosphere of said insulating oil is checked by checking whether said oil-filled electrical equipment is of open type or of sealed type.

5. The diagnosis method according to claim 1, wherein
said byproduct is at least one kind of compound selected from a group consisting of benzaldehyde, benzyl alcohol, bibenzyl, dibenzyl sulfide, and dibenzyl sulphoxide.

6. A maintenance method for oil-filled electrical equipment, for taking prescribed measures in accordance with said risk, based on said risk diagnosed by using the diagnosis method as recited in claim 1.

7. The maintenance method according to claim 6, wherein
said measures include at least any one of addition of the copper sulfide generation inhibitor, recommendation of an oil degradation preventing method for the oil-filled electrical equipment, replacement with new oil that does not contain dibenzyldisulfide, and update of the insulating oil.
